(19) United States
(12) Patent Application Publication
(10) Pub. No.: US 2022/0347457 A1
(43) Pub. Date: Nov. 3, 2022

(54) ABSORBENT CAP FOR ISOLATING AND DEACTIVATING HAZARDOUS FLUID RESIDUES ON MEDICAL CONNECTORS AND METHOD OF USE

(71) Applicants: Michael W. Howlett, Las Vegas, NV (US); James V. Mercer, West Jordan, UT (US); Brad C. Holt, Midvale, UT (US)

(72) Inventors: Michael W. Howlett, Las Vegas, NV (US); James V. Mercer, West Jordan, UT (US); Brad C. Holt, Midvale, UT (US)

(21) Appl. No.: 17/733,475

(22) Filed: Apr. 29, 2022

Related U.S. Application Data
(60) Provisional application No. 63/182,645, filed on Apr. 30, 2021.

(51) Int. Cl. A61M 39/20; A61M 39/16

(52) U.S. Cl. CPC A61M 39/20; A61M 39/162; A61M 2039/1033

(57) ABSTRACT

A cap for medical connectors that absorbs deactivates and isolates hazardous fluid residues on the ends of medical connectors after administration of hazardous fluids.

This application is claims priority to and the benefit of U.S. Provisional Patent Application No. 63/182,645, filed on Apr. 30, 2021, which is hereby incorporated by reference in its entirety.

### BACKGROUND

Infusible hazardous drug (HD) fluids in medical therapy pose a significant risk to the patients, health care workers and other individuals in both inpatient and outpatient treatment facilities. FDA regulations such as USP797 and USP800 have been enacted to require the use of safer devices to reduce HD exposures. These safety devices include closed system transfer devices (CTSD) that help prevent HD fluid from escaping the fluid pathway and entering the external environment. Although CTSDs have greatly reduced HD exposures in the health care setting, problems remain. Once such problem is that a droplet of HD fluid residue may linger at the junction of IV tubing connectors when they are disconnected. This residue may not be visible to the naked eye, which may lull healthcare workers into a false sense of security, believing it is not there. These droplets may contaminate healthcare workers fingers or gloves, or the droplets may be dislodged and “flicked” into the environment during handling. Further, these droplets may vaporize into the air around healthcare workers and others where it can be inhaled.

When healthcare workers observe this HD residue on connectors they may use a gauze pad (or similar) to wipe away the residue. However, HD fluid residue can soak through the gauze and get on fingers or gloves. Furthermore, the gauze may facilitate greater evaporation of the HD fluid into room air and will continue to do so even after it is discarded into HD waste containers that are not airtight.

What is needed is a capping device that can be applied to HD tubing connectors after disconnection that absorbs and deactivates HD fluid residues that remain on HD connectors after use. Also, this capping device needs to be closeable and sealable after use to capture HD residues within the capping device and prevent it from vaporizing into the environment.

### SUMMARY OF THE INVENTION

The disclosure of the present invention relates to a medical fitting capping device or more specifically, to an absorbent closeable cap for HD fluid tubing connectors, particularly those connectors used during administration of chemotherapy.

The present invention, or closeable absorbent cap (the cap) comprises an extended body structure (housing) with a closed end and an internal cavity, the open end of the housing configured to receive a target connector and removably attach to it, the cavity containing dry highly absorbent material hereafter called a “sponge”, the open end of the cap being sealed by a lid closure or plug before use that may remain tethered to the housing when covering a target connector and may be replaced onto the open end of the cap when it is removed from the target connector.

In the first embodiment of the invention, the cap's open end is configured to screw onto a target needleless injection site (NIS) commonly used in infusion therapy in the same fashion as currently used disinfectant caps for NIS. Instead of a fluid-soaked sponge such those used in current disinfectant caps, the invention utilizes a dry, highly absorbent material or sponge that soaks up HD fluid residue that may be present on the end of the NIS when the cap is screwed onto an NIS. The cap is sealed by an airtight closure such as a lid or plug (plug-seal) prior to use to maintain its sterility and prevent ambient vapors from being absorbed into the cap's absorbent sponge. The plug-seal is unremovably attached to the cap's housing by a tether than enables the plug to move a sufficient distance away from the cap opening in order to not impede it being applied to a target NIS. The tethered plug-seal may be replaced back into the opening of the cap after removal from a target NIS in order to retain any absorbed HD fluid within the cap and prevent hazardous vapor from escaping from the re-sealed cap. The plug-seal recreates an airtight seal when replaced in the cap opening to retain HD fluid vapors within the re-sealed cap. Preferably, the absorbent sponge in this and all other embodiments contains or is impregnated with a substance that deactivates hazardous substances. One example of such a deactivating agent is activated carbon, which is currently used to deactivate HD fluid when cleaning up accidental spills. The deactivating component of the sponge, such as carbon, may be impregnated throughout the sponge material, or it may be a separate component or layer underlying the sponge. The face of the sponge receiving the target connector may have a variety of shapes including concave and convex to facilitate contact of the target connector's fluid pathway opening and adjacent face and threads.

In a second embodiment of the invention, the cap's open end is configured to receive a male luer fitting such as an IV tubing end, and particularly a closed male luer (CML) connector commonly used in HD therapy.

In a third embodiment, the cap is configured to receive an NIS on one end and a male-luer connector on the other end. This dual-purpose cap may have a partition in the center to keep the sponge chamber in each side of the cap disparate. This enables a single two-function cap to cover the end of either an NIS (female) connector or a male luer connector.

In a fourth embodiment, the cap may be configured without threads so that a target connector may be inserted into an enlarged internal cavity with a friction fit or snap-on fitting. This embodiment is much like “dust covers” currently used to protect the sterile ends of HD connectors during transport from the pharmacy to the patient care area. It is contemplated that current dust covers may be adapted for use as an absorbent cap by adding an absorbent sponge. This fitted embodiment is typically designed to correspond to a specific brand and make of connector. This fitted cap would contain a sponge at its closed end as with the above embodiments, and preferably would contain a sponge impregnated with a deactivating substance.

In a fifth embodiment, the tethered plug-seal described in the first embodiment may be replaced by seal that is not tethered. Rather, this plug-seal may be removed immediately before it is used to cover a target connector and set aside. Preferably, this plug-seal may be attached to the closed end of the cap, much like how the removable cap of a writing pen can be removed from its tip and attached to the opposite end on the pen for later use covering the pen tip. After the cap is removed from its target connector, this plug-seal can be removed from the base of the cap and re-placed onto the same opening it was removed from in order to seal harmful vapors within the cap. It is contemplated that this cover may be designed to snap onto the used cap so it cannot be inadvertently removed, releasing potentially harmful vapors into the surrounding air.

In a sixth embodiment, is a cap that is not entirely rigid and may be described as a sealable pouch. The opening to the pouch is formed around a deformable material such as bendable wire so that the user can squeeze the opening ends together to open it, much like a slitted coin change purse. A target connector can be inserted into the cap, followed by pressing and bending the deformable opening around the connector to both seal it off from the surrounding environment, and keep the cap attached to the connector. In this embodiment, it is contemplated that an entire connector can be inserted into this elongated cap, removed from the vascular access device, and then sealed within the cap by squeezing the bendable opening together.

In a seventh embodiment, the front end of a cap is fitted with an absorbent sponge as in the above embodiments. The opposite end of the same cap is comprised of a disinfectant cap for application to the same target connector after removal of the absorbent cap. This disinfectant end of the cap having a seal, such as foil, that is of different make, shape and color to distinguish it from the absorbent end of the cap, the seal being removable and not tethered. This embodiment enables the user to use the absorbent end of the cap to remove and isolate HD residue followed by disinfection of the same cap.

In an eight embodiment, the cap may be provided in a specialized packaging format in which at least two caps are packaged in a carrier strip that may be hung from a device such as an IV pole boom. Caps may be fitted into a hole in a strip prior to the tethered plug-seal being wrapped over the edge of the strip and sealing onto the top opening of the cap positioned through the hole. The tethered plug-seal has a wider diameter than the hole in the strip so that a sealed cap cannot slip out of the bottom side of the strip. The hole is sized to fit over the opening rim of the cap, but smaller than the diameter of the tri-lobed housing so that a sealed cap cannot slip out of the top of the strip. To remove a cap from the strip, the user removes and lifts the tethered plug-seal off the cap opening then grasps the cap housing underneath the strip and pulls the untethered cap out through the bottom of the strip. The loop in the plug-seal's tether further keeps the cap from slipping out of the strip unintentionally. The strip may be slitted or scored where the loop of the tether wraps around the strip so that this slit deforms or tears apart so that the loop and cap can be pulled off of the strip without removing the plug seal. This may be needed if the cap is not going to be used immediately.

### BRIEF DESCRIPTION OF DRAWINGS

FIG. 1A top perspective view of the invention, an absorbent cap with tri-lobed housing

FIG. 2A bottom perspective view of an absorbent cap

FIG. 3A perspective view of an absorbent sponge layer

FIG. 4A perspective view of a deactivating (e.g. carbon) sponge layer

FIG. 5A perspective view of a combined or consolidated absorbent and deactivating sponge

FIG. 6A top and bottom perspective view of a concave tethered lid closure or plug-seal

FIG. 7A top perspective view of an absorbent cap and attached lid closure

FIG. 8A bottom perspective view of an absorbent cap with an attached lid closure and an image of the standard biohazard symbol embossed on the bottom of the cap

FIG. 9A cross-sectional view of an absorbent cap, tethered lid closure, threads for receiving a medical connector, and absorbent sponge layers within the cap's internal cavity

FIG. 9A An exploded view of (from left to right) a medical connector, sponge layers, and absorbent cap with a tethered lid closure

FIG. 10A cross-sectional view of a medical connector inserted into an absorbent cap

FIG. 11A cross-sectional view of an absorbent cap for male luer connectors

FIG. 12A side perspective view of a male luer connector being received by an absorbent cap

FIG. 13A cross-sectional view of an absorbent cap with a non-tethered screw-in lid closure that may also be screwed into the bottom end of the cap until it is used to seal the top end of the same cap after use

FIG. 14 Perspective views of FIG. 13

FIG. 15A cross-sectional view of an absorbent cap with a non-tethered, friction-fit lid closure that may also be friction-fitted on the bottom end of the cap until it is used to seal the top end of the same cap after use

FIG. 16A cross-sectional view of an absorbent cap as is FIG. 15 with a friction-fit lid that utilizes a snap-on latch to retain the lid closure

FIG. 17 Perspective view of an absorbent cap with a tethered lid closure that friction-fits onto a medical connector without screwing onto it

FIG. 18A cross-sectional view of FIG. 17 showing a medical connector being capped

FIG. 19A cross-sectional view of an absorbent cap in the left side, and on the right side a fitting for receiving and disinfecting a medical connector with antiseptic fluid

FIG. 20A top perspective view of FIG. 19

FIG. 20A A bottom perspective view of FIG. 19

FIG. 21A perspective view of an absorbent cap with a convex sponge configured to receive a medical connector with a recessed fluid pathway fitting

FIG. 22A cross-sectional view of FIG. 21

FIG. 23A perspective view of a sponge-lined closeable pouch for receiving and isolating a used medical connector

FIG. 24A perspective view of a hangable carrier strip for packaging absorbent caps that remain anchored to the strip using the tethered lid closure

### DETAILED DESCRIPTION

The following description of the invention can be understood in light of the Figures which illustrate specific aspects of the embodiments and are part of the specification. Together with the following description, the Figures demonstrate and explain the principles of the invention. In the Figures, the physical dimensions of the embodiment may be exaggerated for clarity. The same reference numerals or word descriptions in different drawings represent the same or similar element, and thus their descriptions may be omitted.

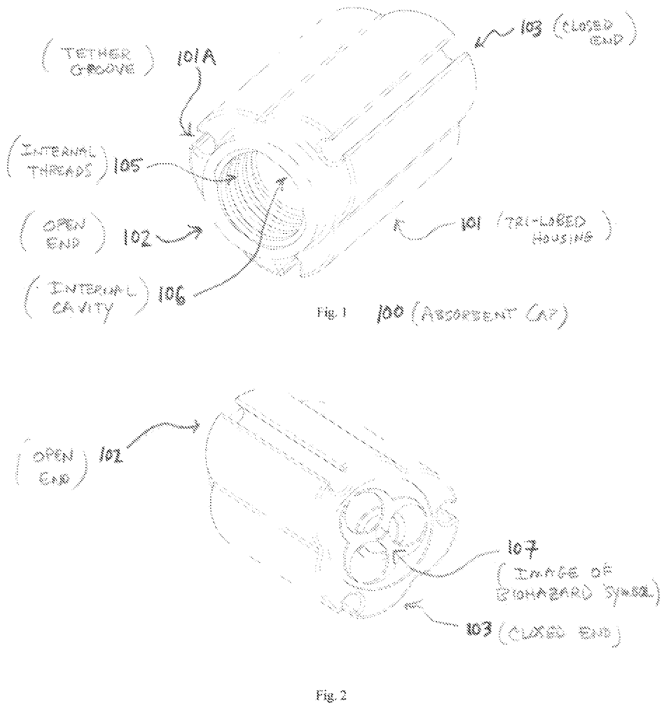

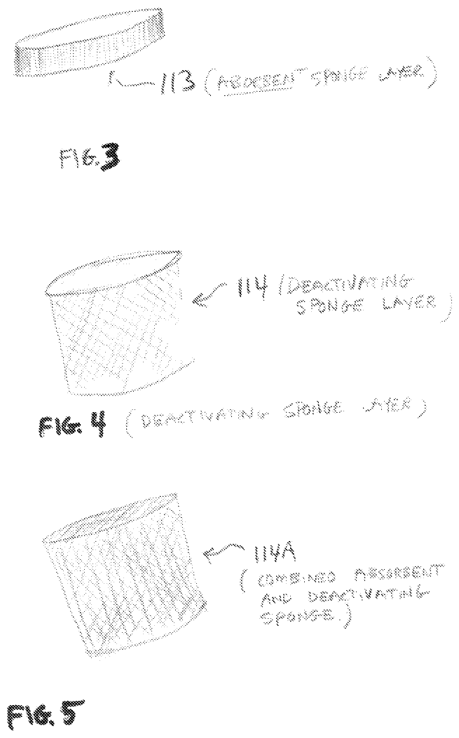

The present invention or absorbent cap 100, as described in FIGS. 1 and 2, including a closed end 103 and an open end 102, an extended body portion or housing formed in a tri-lobe shape 101 comprising a rigid internal cavity 106 formed in the bottom portion of the housing, internal threads 105 formed inside the opening of the housing to receive the corresponding threads of a medical connector, and semi-circular longitudinal grooves 101A in the apex of the three housing lobes. The bottom outside surface of the housing 103 formed or colored with the image of the standard biohazard symbol 107 to convey this hazard to the user.

FIGS. 3-5 show absorbent sponges placed in layers in the internal cavity 106 of the cap. The internal cavity of the cap containing a thinner absorbent sponge layer 113 closest to the cap opening, and a thicker deactivating sponge layer 114 underneath it. This is to prevent the deactivating material in the second sponge from directly contacting the fluid pathway opening. Preferably, the absorbent and deactivating components of the two sponge layers may be consolidated into a single sponge 114A having both properties.

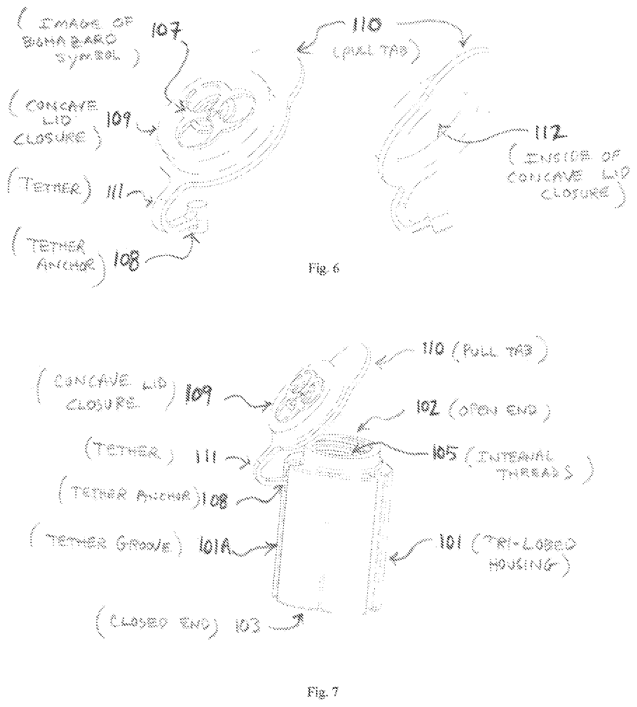

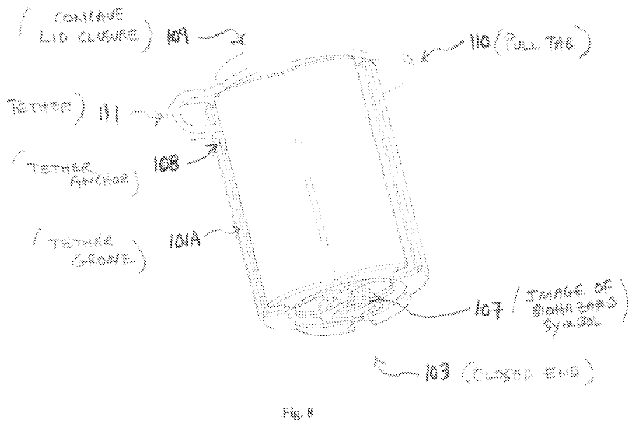

FIGS. 6, 7 and 8 show the absorbent cap 100 covered and sealed by a concave lid closure 109 or plug-seal that utilizes a tether 111 with a tether anchor 108 on its distal end that fits into the tether groove 101A in the cap housing. The tether anchor may be designed to slide freely within the tether groove enabling the plug-seal to be moved further from the cap opening if desired. The plug-seal may have an embossed or colored image of the biohazard symbol 107 on its external top surface, thus providing this warning symbol on both the bottom and top of the cap. The lid closure or plug-seal has an elongated portion that acts as a pull-tab 110. This tab may also be comprised of material sufficiently rigid to facilitate the plug-seal being removed by pushing up on the tab with a finger or thumb. This is to facilitate one-handed removal of the plug-seal.

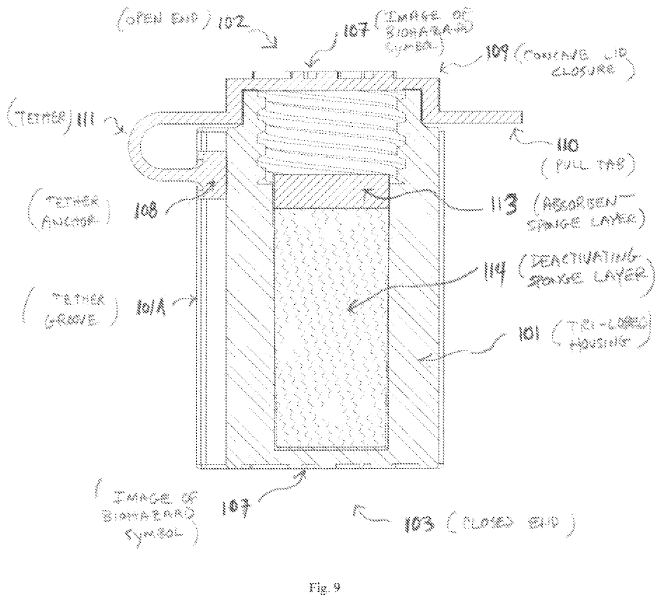

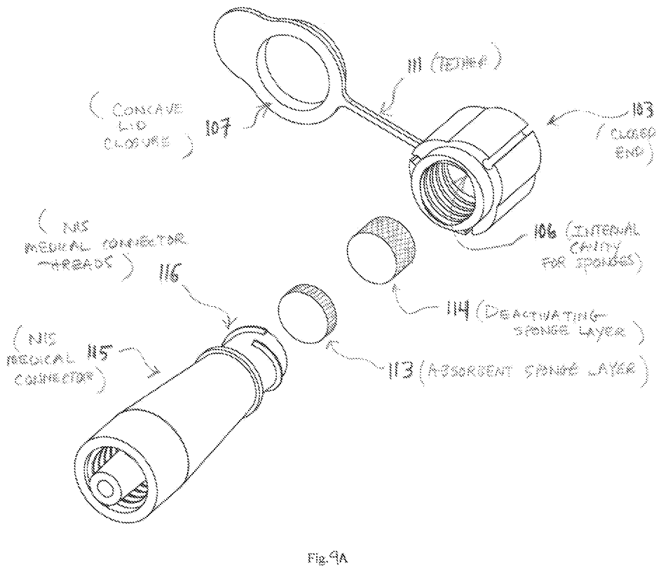

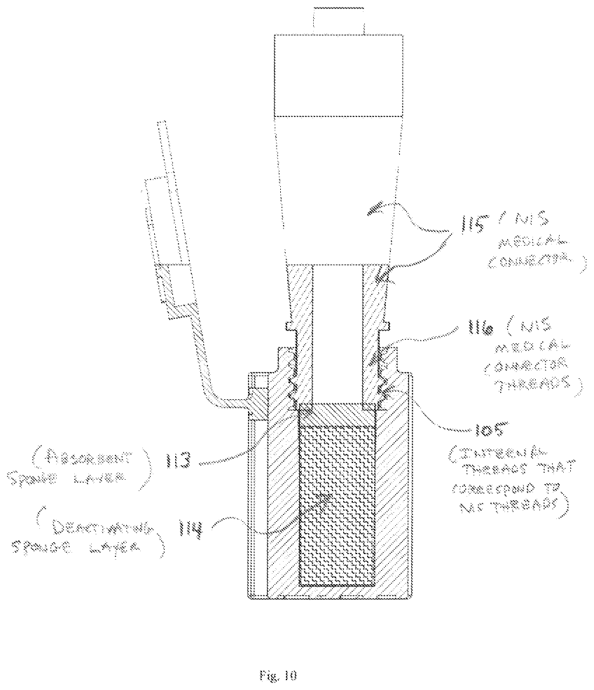

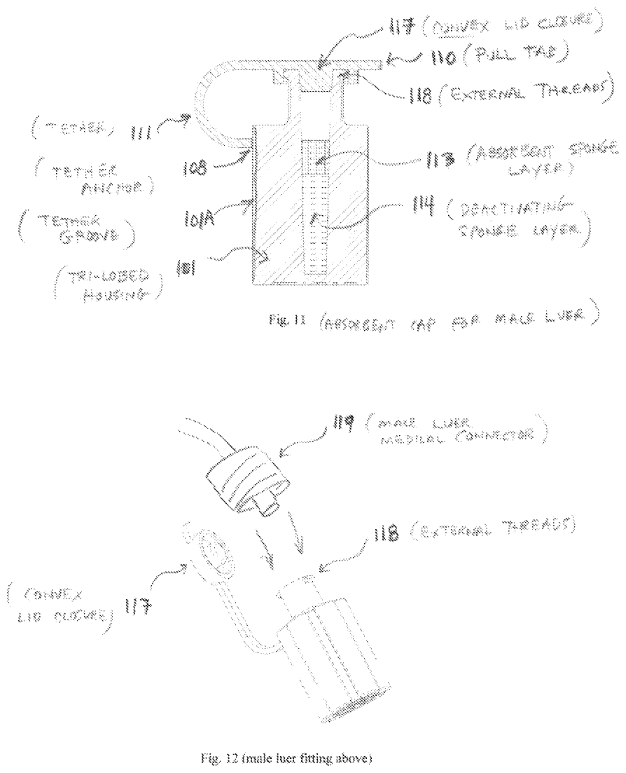

FIG. 9 shows the various components of the cap in a cross-sectional view including the internal threads 105 in the opening of the cap for receiving a medical connector such as a needleless injection site (NIS). FIG. 9A is an exploded view of the components of the cap showing how the absorbent sponges 113 and 114 fit into the internal cavity 106 of the cap. A medical connector 115 is shown in a position to be received by the cap. FIG. 10 shows a medical connector threads 116 screwed into the cap's opening threads 105. The face of the medical connector is shown in contact with the absorbent sponge so that it can soak up any HD fluid residue at the fluid pathway opening of the medical connector.

FIG. 11 shows an absorbent cap for use on male luer medical connectors with external threads 118 at the opening, rather than internal threads. The plug-seal or lid closure is formed in a convex shape 117 to fit inside the opening of this male luer cap rather than over the outside rim of the NIS cap. The other components of the male luer cap are identical to the first embodiment.

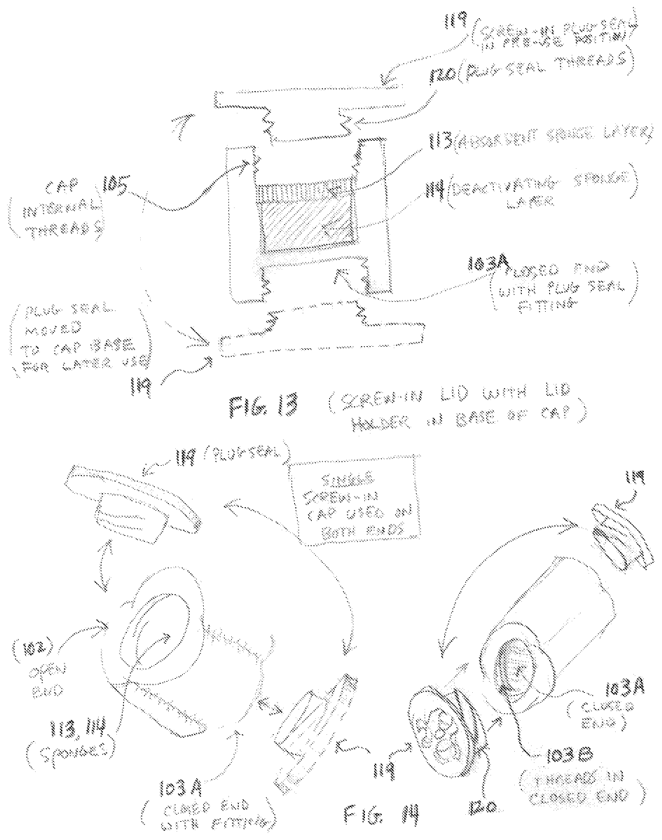

FIGS. 13 and 14 shows a cap without a tethered plug-seal. Instead, a non-tethered plug-seal 119 with threads 120 screws into the corresponding internal threads 105 of the open end of the cap. This plug-seal may be removed and screwed into the closed bottom end 103A of the same cap that also has corresponding threads so that the plug-seal can be attached there until after the cap is used on a target medical connector. Once the absorbent end of the cap is removed from a target medical connector, the screw-in cap 119 can be removed from the bottom fitting of the cap 103 and re-placed on the open end of the cap 102 to seal absorbed HD fluid and vapors within it.

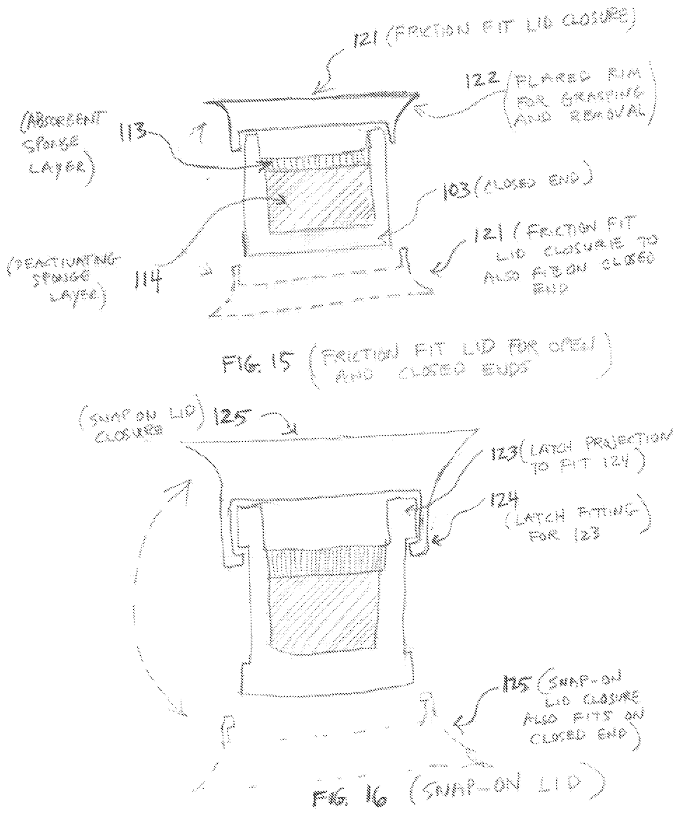

FIG. 15 shows an untethered lid closure or plug-seal that friction-fits 121 onto the cap rather than screw into it. This plug-seal has a flared top 122 to aid in grasping and removal. This plug-seal 122 can also be fitted onto the bottom of the cap 103 for later use as described above.

FIG. 16 shows a non-tethered lid closure or plug-seal as in FIG. 15 that has a snap-on latch 123 and corresponds to a latch on the housing of the cap 124. This snap-on latch provides a more secure attachment for the plug-seal that friction fit.

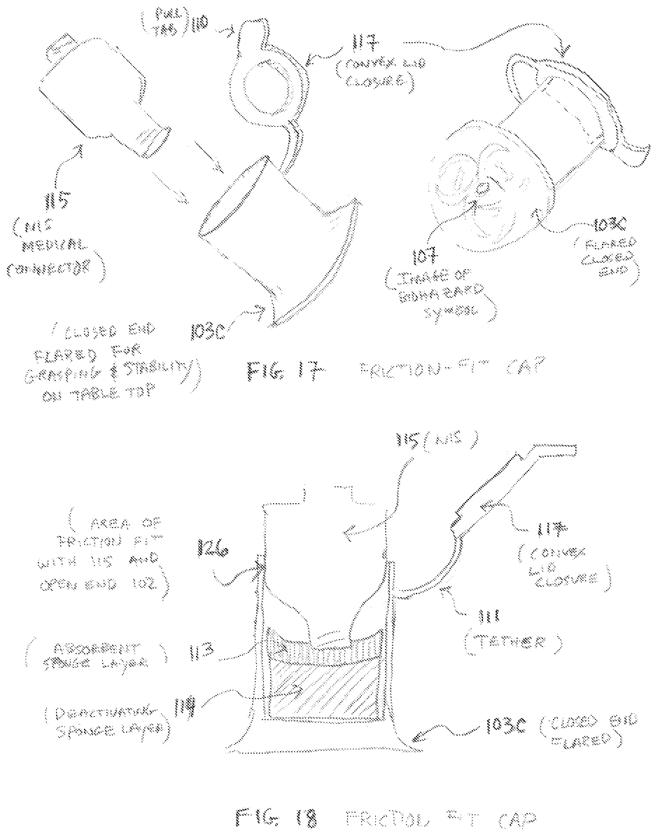

FIG. 17 shows a cap with a tethered plug-seal that friction fits onto a medical connector rather than screws onto it. This embodiment has no threads in its opening and is typically formed to fit a specific connector. The area of friction fit 126 may be on the housing of the medical connectors rather than its narrower, threaded front end. The fluid pathway interface of the medical connector touches the absorbent sponges 113, 114 in the cap as with other embodiments.

FIG. 19 shows where droplets of HD fluid 127 may reside after medical connectors are separated.

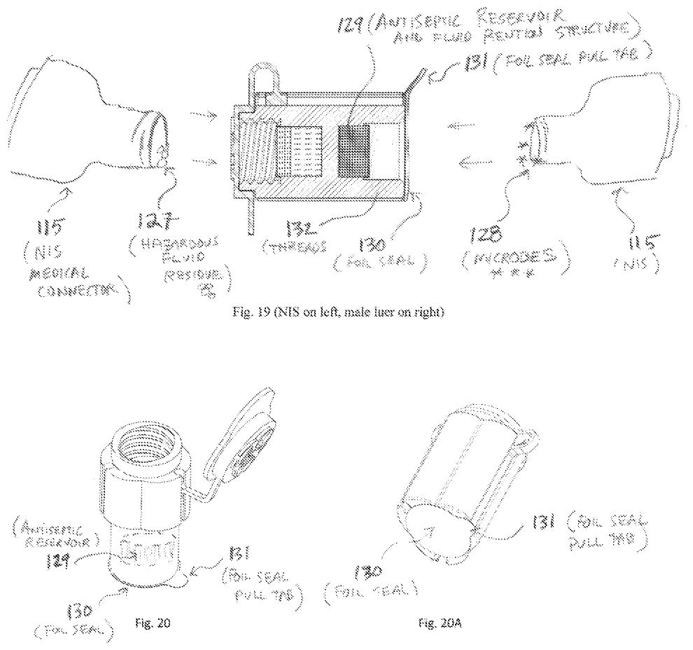

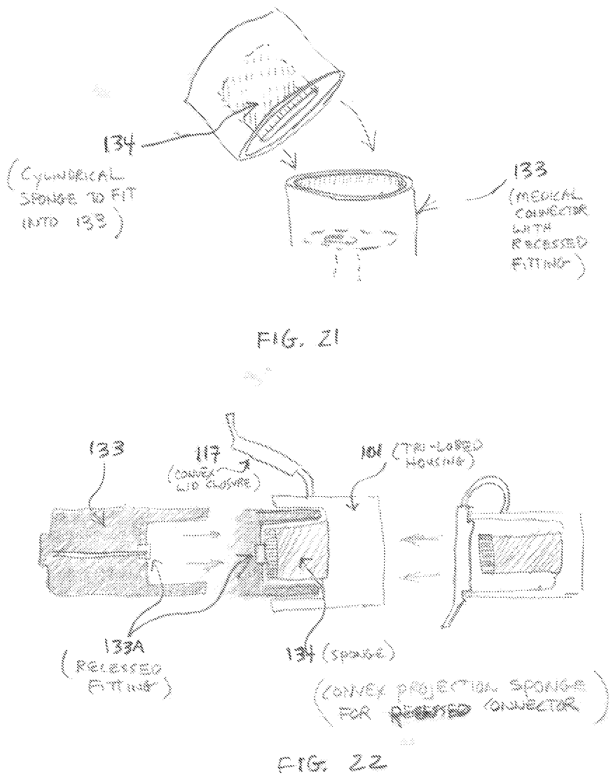

FIGS. 19, 20 and 20A show a dual-purpose absorbent cap. One end of the cap is absorbent as in FIGS. 1-10. The other end of the same cap is not closed and is formed to comprise an internal antiseptic reservoir and fluid retention structure such as an alcohol-impregnated sponge 129 for the purpose of receiving and applying disinfectant such as isopropyl alcohol to a medical connector to kill and microbes 128 dwelling thereon. The disinfectant reservoir is covered by a seal 130 with a pull tab 131 or similar to retain disinfectant therein, this seal, such as a foil seal, being substantially different in make and color to distinguish it from the plug-seal on the opposite, absorbent end of the dual-purpose cap. This seal is NOT reusable as are plug-seals 119, 121 in the absorbent cap embodiments. It is contemplated that such a dual-purpose cap may also be constructed with absorbent caps on both ends that may fit disparate connectors such an NIS cap on one end and a male luer cap on the other.

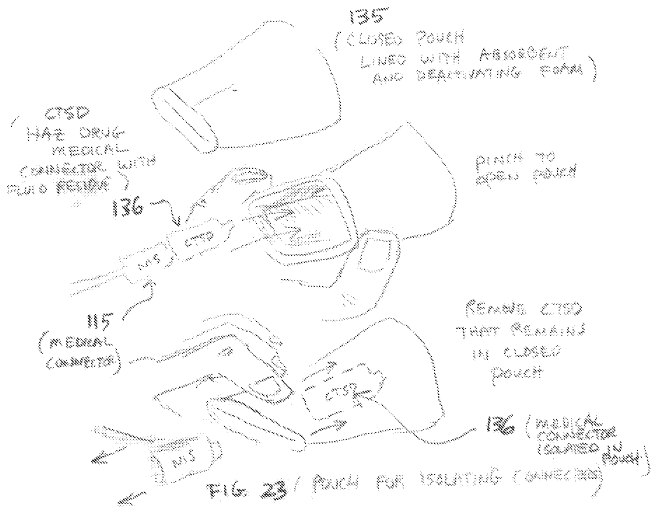

FIGS. 21 and 22 show a specialized absorbent cap with a cylindrical absorbent sponge 134 that may be inserted into a medical connector with a recessed fluid pathway fitting 133. This is needed to assure that the absorbent sponge contacts the recessed fitting and associated fluid pathway opening.

FIG. 23 discloses a closeable pouch 135 for receiving and isolating a medical connector 136 that may contain or be contaminated with hazard fluid. The pouch is lined with an absorbent sponge as in items 113, 114 and has an opening constructed with a deformable material such as a bendable aluminum strip that can be opened by pinching the sides together and closed by squeezing the opening shut.

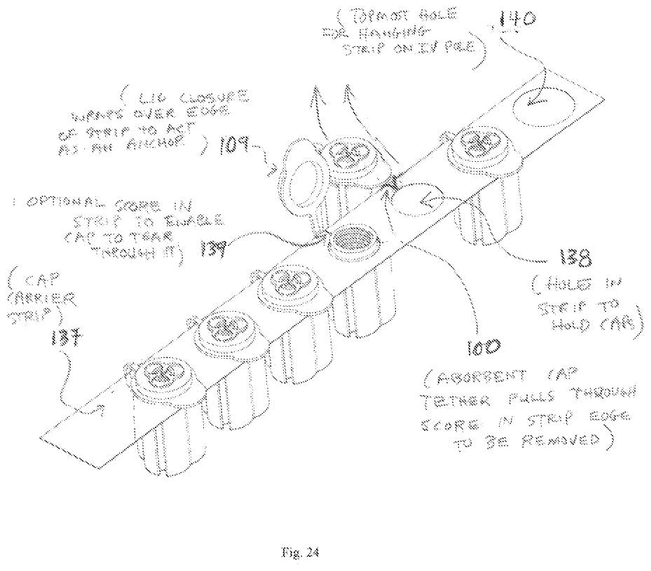

FIG. 24 shows a carrier strip 137 for absorbent caps with holes 138 configured so that the opening of an unsealed cap fits through the hole, but not the wider tri-lobed body of the cap. The tethered plug-seal 109 can then be wrapped around the edge of the strip and sealed onto the cap opening protruding through the hole in the strip. The plug-seal, having a wider diameter than the hole, prevents the cap from sliding out of the bottom of the hole. Sealed caps therefore are anchored to the strip and cannot be removed until the plug-seal is lifted out of the way, whereupon the cap can be grasped from the bottom and pulled out the bottom of the strip. Alternatively, the carrier strip can be scored or perforated 139 near the tether of the plug-seal to enable caps to be torn out of the strip without removing the plug-seal. The carrier strip has at least one hole or aperture 140 in its top end for hanging the strip on a hook or IV pole boom.

1. A medical connector cap comprising,
a housing with a closed end and an open end at opposite ends of the housing in a longitudinal direction, the housing including:
an internal cavity located in the housing proximate the open end of the housing,
a connector receiver proximate the open end of the housing, and
at least one absorbent material contained in the internal cavity.

2. The medical connector cap of claim 1, the internal cavity also containing at least one substance that deactivates hazardous fluid.

3. The medical connector cap of claim 1, further comprising a closure that selectively seals the open end of the housing.

4. The medical connector cap of claim 3, the closure having an image warning that the device is used to absorb hazardous fluid.

5. The medical connector cap of claim 3, wherein the closure is tethered to the housing.

6. The medical connector cap of claim 3, wherein the closed end and the open end are configured to selectively receive and couple to the closure to cover the closed end and open end, respectively.

7. The medical connector cap of claim 3, wherein the closure complementarily mates with the connector receiver of the open end.

8. The medical connector cap of claim 1, wherein the connector receiver includes internal threads.

9. The medical connector cap of claim 1, wherein the connector receiver includes a male luer connection.

10. The medical connector cap of claim 1, in which the closed end of the cap comprises a cavity containing a fluid reservoir for a disinfectant, the reservoir having a structure for retaining fluid in the reservoir, and an opening configured to receive a target medical connector, and a removable seal for the disinfecting receiver to prevent disinfectant fluid from escaping from the reservoir.

11. The medical connector cap of claim 1, further comprising a bright yellow color to signal that it is used to absorb hazardous fluid and therefore may also be hazardous.

12. The medical connector cap of claim 1, the housing having a trilobed shape similar to the biohazard symbol to signal it is used to absorb hazardous fluid and therefore may be hazardous.

13. The medical connector cap of claim 1, wherein the least one absorbent material is a substance that deactivates hazardous fluid.

14. A medical connector cap system, the system comprising:
a plurality of medical connector caps, each of the medical connector caps of the plurality of medical connector caps including:
a housing with a closed end and an open end at opposite ends of the housing in a longitudinal direction, the housing including:
an internal cavity located in the housing proximate the open end of the housing,
a connector receiver proximate the open end of the housing,
at least one absorbent material contained in the internal cavity, and
a closure connected to the housing by a closure tether; and
a carrying device including a strip of material with a series of holes configured to fit and hold the housing of each medical connector cap in a fixed position, and the closure being coupled to the housing such that the tether is attached to the housing at a first end underneath the strip and the closure is coupled to a second end with the strip of material captured in a tether loop formed by the tether

15. The carrying device in claim 14 with the strip being scored or perforated near the tether loop to enable the tether loop and cap to tear through the score in the strip and thereby enable the cap to be separated from the strip without decoupling the closure.

16. A medical connector cap comprising,
a housing with a first end and a second end at opposite ends of the housing in a longitudinal direction, the housing including:
a first internal cavity located in the housing proximate the first end of the housing,
a second internal cavity located in the housing proximate the first end of the housing,
a first connector receiver proximate the first end of the housing,
a second connector receiver proximate the second end of the housing, the second connector receiver being different from the first connector receiver,
at least one absorbent material contained in the first internal cavity, and
at least one absorbent material contained in the second internal cavity.

17. The medical connector cap of claim 16, wherein at least one of the first internal cavity and the second internal cavity includes a deactivating material.

18. The medical connector cap of claim 16, wherein at least one of the first internal cavity and the second internal cavity contains a fluid reservoir for a disinfectant and a removable seal for the disinfecting receiver to prevent disinfectant fluid from escaping from the reservoir.

19. The medical connector cap of claim 18, wherein the fluid reservoir has an opening configured to receive a target medical connector.

20. The medical connector cap of claim 16, wherein the first connector receiver includes internal threads and the second connector receiver includes a male luer connection.
